(19) United States
(12) Patent Application Publication
(10) Pub. No.: US 2006/0251706 A1
(43) Pub. Date: Nov. 9, 2006

(54) Multi purpose cartridge system for delivery of beneficial agents

(76) Inventor: Imre Virag, Toronto (CA)

Correspondence Address: Imre Virag, 220 Lake Promenade #718, Toronto, M8W 1A9, CA

(21) Appl. No.: 11/183,098

(22) Filed: Jul. 18, 2005

(30) Foreign Application Priority Data
May 4, 2005 (CA) 2,503,456

(51) Int. Cl. A61F 13/02; A61L 15/16

(52) U.S. Cl. 424/448

(57) ABSTRACT

This design provides an effective means of delivering to specific areas of the human or animal body the beneficial effects of substances such as minerals, quartz or salts natural or artificial thermal agents other agents used to provide temporary or permanent relief The cartridge is composed of two different types of film. The layer of the cartridge applied towards the body is made from a transparent film such as transparent bio-oriented polypropylene to offer free flow of the perceived benefits of the substances to the designated body part. The layer of film on the side of the cartridge that is away from the body is made of metallic film such as metallized oriented polypropylene to allow the reflection of the perceived substance's benefits onto the body surface. Cartridges are located in garments or health appliances.

The cartridge is deployed in a pocket or array of pockets. Each cartridge is formed by sealing a measured quantity of the desired substance between two layers of film.

Each cartridge is strong enough to safely and securely hold the desired substance for delivery to the specific area of the body, and flexible enough to follow the contour of the body parts to which they are applied.

The cartridge is composed of two different types of film. The side of the cartridge applied towards the body is made from a transparent film such as transparent asymmetrical bi-oriented polypropylene film which allows free flow (of the perceived benefits of the substances) to the designated body parts. The side of the cartridge which is away from the body, is made of metallic film such as metallized oriented polypropylene in order to allow the reflection of the perceived substance's benefits onto the body surface. The film on the outer side of the cartridge also acts as a type of insulation.

The size of the cartridge will depend on the desired use, the volume of substance to be delivered, and location on the body.

The cartridge delivery system can be sewn, or glued, or attached by other means onto body joint support systems, or undergarments, tee-shirts, active wear garments or health appliances.

FIG. ONE shows a typical cartridge of the system of a size that often used.

FIG. TWO shows an array of the cartridges deployed on a wrist support system designed by the inventor Imre Virag, and supplied by Standing River Incorporated of Canada.

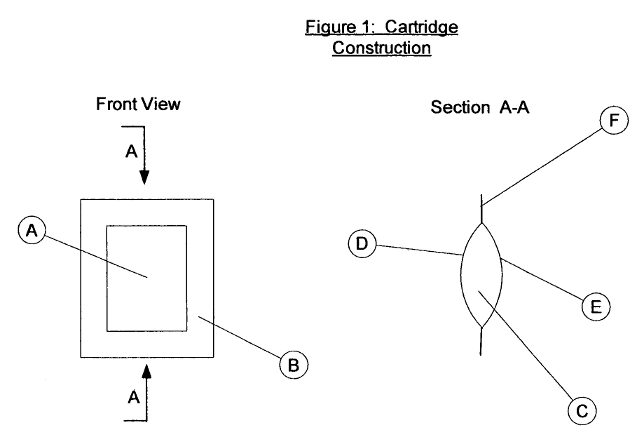

FIG. 1 shows the construction of the Cartridge.

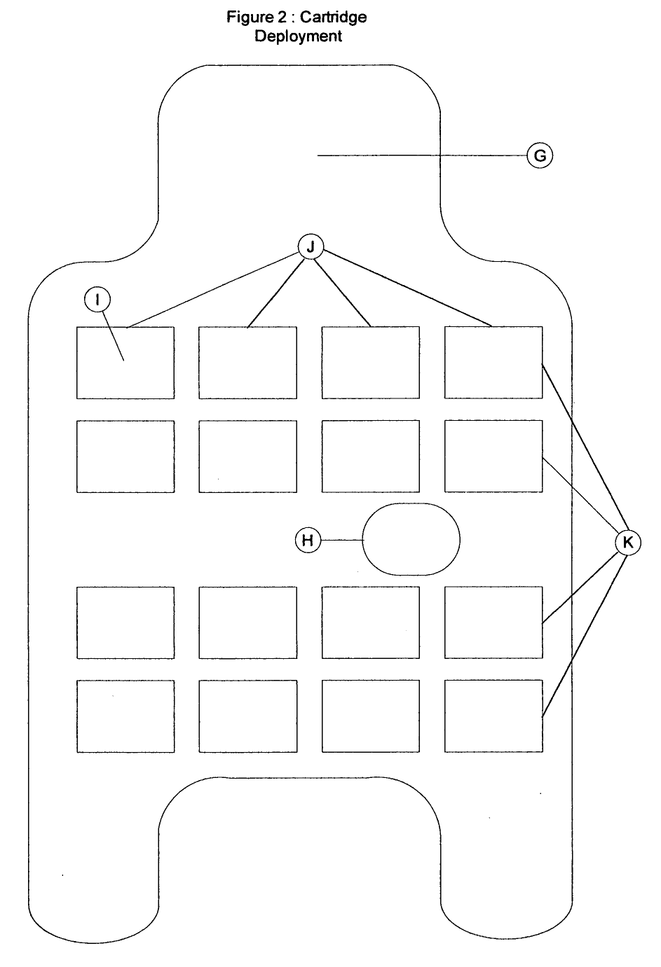

A. The cartridge contains the proposed beneficial substance contained in a pocket between the two films that are used to construct the pocket. For a small pocket the typical dimensions would be approximately ¾″ by 1″
B. Typically a ¼″ seam allowance around beneficial substance to form seal between two film components. The seal is created by heat or other methods of attachment.
C. The beneficial substance between the two sealed film components is shown in section A-A.
D. The translucent polypropylene film which is applied toward the body.
E. The metallized polypropylene film is applied away from the body.
F. The two films are sealed together at the edges.
   FIG. 2 shows a typical deployment of the cartridges in a joint support used on the wrist.
L. The Wrist Support is made of a material such as Neoprene, and deploys in this case 16 Cartridges deployed in 4 arrays.
M. This is merely the hole in the support for the Thumb, and forms no part of the deployment capability.
N. A single cartridge as described in FIG. 1 above.

O. Shows an array of 4 cartridges aligned in the horizontal direction.
P. Shows an array of 16 cartridges aligned in both the horizontal and vertical directions.

What is claimed is:

1. This design provides an effective means of delivering to specific areas of the human or animal body the beneficial effects of the following substances:
minerals, quartz or salts.
natural and artificial thermal agents.
other agents used to provide temporary or permanent relief.

2. It is not the intention of this patent request to support or refute the health benefits of the above mentioned substances.

3. The intent of this patent request is to offer a convenient and economical delivery system consisting of the cartridge and pockets for the deployment of the above mentioned substances allowing them to affect the specific area of the body to which they are applied.

4. The cartridge delivery system can be utilized in combination with any garment, such as undergarments, tee-shirts, active wear garments, body joint supports such as “Tensor” products as well as body joint supports such as wrist, elbow, shoulder, knee, and ankle supports designed by other companies such as Standing River of Canada.

5. Each cartridge is strong enough to safely and securely hold the desired substances allowing delivery of the substance's benefits to the specific area of the body.

6. Each cartridge is flexible enough to follow the contour of the body parts to which it is applied.
